SEQUENCE LISTING (101 sequences)

SEQ ID NO: 1 - protein, 9 aa, Homo sapiens
AAFPGASLY

SEQ ID NO: 2 - protein, 9 aa, Homo sapiens
AELATRALP

SEQ ID NO: 3 - protein, 9 aa, Homo sapiens
AFFAERLYY

SEQ ID NO: 4 - protein, 9 aa, Homo sapiens
ALATLIHQV

SEQ ID NO: 5 - protein, 9 aa, Homo sapiens
ALAVIITSY

SEQ ID NO: 6 - protein, 9 aa, Homo sapiens
ALQEMVHQV

SEQ ID NO: 7 - protein, 9 aa, Homo sapiens
ALRDVRQQY

SEQ ID NO: 8 - protein, 9 aa, Homo sapiens
AQNAVRLHY

SEQ ID NO: 9 - protein, 9 aa, Homo sapiens
AQPGFFDRF

SEQ ID NO: 10 - protein, 9 aa, Homo sapiens
AVCEVALDY

SEQ ID NO: 11 - protein, 9 aa, Homo sapiens
AVLGAVVAV

SEQ ID NO: 12 - protein, 10 aa, Homo sapiens
EEHPTLLTEA

SEQ ID NO: 13 - protein, 10 aa, Homo sapiens
EEHPTLLTEA

SEQ ID NO: 14 - protein, 9 aa, Homo sapiens
EEMPQVHTP

SEQ ID NO: 15 - protein, 8 aa, Homo sapiens
EENFAVEA

SEQ ID NO: 16 - protein, 9 aa, Homo sapiens
EENKLIYTP

SEQ ID NO: 17 - protein, 9 aa, Homo sapiens
FAEGFVRAL

SEQ ID NO: 18 - protein, 9 aa, Homo sapiens
FFGETSHNY

SEQ ID NO: 19 - protein, 9 aa, Homo sapiens
FLPHMAYTY

SEQ ID NO: 20 - protein, 10 aa, Homo sapiens
GEPRFISVGY

SEQ ID NO: 21 - protein, 9 aa, Homo sapiens
GLATDVQTV

SEQ ID NO: 22 - protein, 9 aa, Homo sapiens
GLNDETYGY

SEQ ID NO: 23 - protein, 9 aa, Homo sapiens
GQEFIRVGY

SEQ ID NO: 24 - protein, 9 aa, Homo sapiens
GQFPGHNEF

SEQ ID NO: 25 - protein, 9 aa, Homo sapiens
GQPWVSVTV

SEQ ID NO: 26 - protein, 9 aa, Homo sapiens
GYLHDFLKY

SEQ ID NO: 27 - protein, 9 aa, Homo sapiens
HQITVLHVY

SEQ ID NO: 28 - protein, 9 aa, Homo sapiens
HVIDVKFLY

SEQ ID NO: 29 - protein, 9 aa, Homo sapiens
HVNDLFLQY

SEQ ID NO: 30 - protein, 9 aa, Homo sapiens
IAMATVTAL

SEQ ID NO: 31 - protein, 9 aa, Homo sapiens
IGIDLGTTY

SEQ ID NO: 32 - protein, 9 aa, Homo sapiens
ILHDDEVTV

SEQ ID NO: 33 - protein, 9 aa, Homo sapiens
IQKESTLHL

SEQ ID NO: 34 - protein, 8 aa, Homo sapiens
ISRELYEY

SEQ ID NO: 35 - protein, 9 aa, Homo sapiens
KLHGVNINV

SEQ ID NO: 36 - protein, 9 aa, Homo sapiens
KQMEQVAQF

SEQ ID NO: 37 - protein, 9 aa, Homo sapiens
KVADMALHY

SEQ ID NO: 38 - protein, 9 aa, Homo sapiens
LEEDSAREI

SEQ ID NO: 39 - protein, 9 aa, Homo sapiens
LLAERDLYL

SEQ ID NO: 40 - protein, 9 aa, Homo sapiens
LLDEEISRV

SEQ ID NO: 41 - protein, 9 aa, Homo sapiens
LLYPTEITV

SEQ ID NO: 42 - protein, 9 aa, Homo sapiens
LMDHTIPEV

SEQ ID NO: 43 - protein, 9 aa, Homo sapiens
LQHPDVAAY

SEQ ID NO: 44 - protein, 9 aa, Homo sapiens
MEDIKILIA

SEQ ID NO: 45 - protein, 9 aa, Homo sapiens
MEENFAVEA

SEQ ID NO: 46 - protein, 8 aa, Homo sapiens
MQKEITAL

SEQ ID NO: 47 - protein, 9 aa, Homo sapiens
NEDLRSWTA

SEQ ID NO: 48 - protein, 9 aa, Homo sapiens
NEIKDSVVA

SEQ ID NO: 49 - protein, 9 aa, Homo sapiens
NVTQVRAFY

SEQ ID NO: 50 - protein, 9 aa, Homo sapiens
NYIDKVRFL

SEQ ID NO: 51 - protein, 10 aa, Homo sapiens
PTQELGLPAY

SEQ ID NO: 52 - protein, 9 aa, Homo sapiens
QEQSFVIRA

SEQ ID NO: 53 - protein, 9 aa, Homo sapiens
QQKLSRLQY

SEQ ID NO: 54 - protein, 9 aa, Homo sapiens
QVAEIVSKY

SEQ ID NO: 55 - protein, 9 aa, Homo sapiens
REHAPFLVA

SEQ ID NO: 56 - protein, 11 aa, Homo sapiens
RLAAAAAQSVY

SEQ ID NO: 57 - protein, 9 aa, Homo sapiens
RLASYLDKV

SEQ ID NO: 58 - protein, 9 aa, Homo sapiens
RNADVFLKY

SEQ ID NO: 59 - protein, 9 aa, Homo sapiens
RQGFVPAAY

SEQ ID NO: 60 - protein, 10 aa, Homo sapiens
RVIEEAKTAF

SEQ ID NO: 61 - protein, 9 aa, Homo sapiens
RVQPKVTVY

SEQ ID NO: 62 - protein, 9 aa, Homo sapiens
RVYPEVTVY

SEQ ID NO: 63 - protein, 7 aa, Homo sapiens
SDHHIYL

SEQ ID NO: 64 - protein, 9 aa, Homo sapiens
SHAILEALA

SEQ ID NO: 65 - protein, 9 aa, Homo sapiens
SISGVTAAY

SEQ ID NO: 66 - protein, 8 aa, Homo sapiens
SPVYVGRV

SEQ ID NO: 67 - protein, 9 aa, Homo sapiens
SWNNHSYLY

SEQ ID NO: 68 - protein, 9 aa, Homo sapiens
SWNNHSYLY

SEQ ID NO: 69 - protein, 9 aa, Homo sapiens
TFMDHVLRY

SEQ ID NO: 70 - protein, 9 aa, Homo sapiens
TLADLVHHV

SEQ ID NO: 71 - protein, 10 aa, Homo sapiens
TLGALTVIDV

SEQ ID NO: 72 - protein, 9 aa, Homo sapiens
TQMPDPKTF

SEQ ID NO: 73 - protein, 9 aa, Homo sapiens
VEHPSLTSP

SEQ ID NO: 74 - protein, 9 aa, Homo sapiens
VEPDHFKVA

SEQ ID NO: 75 - protein, 8 aa, Homo sapiens
VEREVEQV

SEQ ID NO: 76 - protein, 13 aa, Homo sapiens
VFIGTGATGATLY

SEQ ID NO: 77 - protein, 9 aa, Homo sapiens
VLREIAEEY

SEQ ID NO: 78 - protein, 10 aa, Homo sapiens
VLSLLSSVAL

SEQ ID NO: 79 - protein, 9 aa, Homo sapiens
VLYDRVLKY

SEQ ID NO: 80 - protein, 9 aa, Homo sapiens
VMDSKIVQV

SEQ ID NO: 81 - protein, 8 aa, Homo sapiens
VQRTLMAL

SEQ ID NO: 82 - protein, 10 aa, Homo sapiens
YFEYIEENKY

SEQ ID NO: 83 - protein, 9 aa, Homo sapiens
YIFKERESF

SEQ ID NO: 84 - protein, 9 aa, Homo sapiens
YVYEYPSRY

SEQ ID NO: 85 - protein, 10 aa, Homo sapiens
YYRYPTGESY

SEQ ID NO: 86 - protein, 9 aa, Homo sapiens
YYSNKAYQY

SEQ ID NO: 87 - protein, 9 aa, Homo sapiens
SSLPTQLFK

SEQ ID NO: 88 - protein, 9 aa, Homo sapiens
ATFPDTLTY

SEQ ID NO: 89 - protein, 10 aa, Homo sapiens
SIFDGRVVAK

SEQ ID NO: 90 - protein, 9 aa, Homo sapiens
FRFENVNGY

SEQ ID NO: 91 - protein, 10 aa, Homo sapiens
QRYGFSAVGF

SEQ ID NO: 92 - protein, 10 aa, Homo sapiens
ARLSLTYERL

SEQ ID NO: 93 - protein, 9 aa, Homo sapiens
GRYQVSWSL

SEQ ID NO: 94 - protein, 9 aa, Homo sapiens
KRFDDKYTL

SEQ ID NO: 95 - protein, 9 aa, Homo sapiens
TRWNKIVLK

SEQ ID NO: 96 - protein, 9 aa, Homo sapiens
LRFDGALNV

SEQ ID NO: 97 - protein, 9 aa, Homo sapiens
ARFSGNLLV

SEQ ID NO: 98 - protein, 9 aa, Homo sapiens
NRIKFVIKR

SEQ ID NO: 99 - protein, 9 aa, Homo sapiens
GRVFIIKSY

SEQ ID NO: 100 - protein, 9 aa, Homo sapiens
SRFGNAFHL

SEQ ID NO: 101 - protein, 9 aa, Homo sapiens
GRTGGSWFK

(19) United States
(12) Patent Application Publication
(10) Pub. No.: US 2011/0070253 A1
(43) Pub. Date: Mar. 24, 2011

(54) TUMOUR-ASSOCIATED PEPTIDES BINDING TO MHC MOLECULES

(75) Inventors: Hans Georg RAMMENSEE, Tuebingen (DE); Stefan Stevanovic, Tuebingen (DE); Toni Weinschenk, Aichwald (DE); Claudia Trautwein, Wuelfrath (DE); Jörn Dengjel, Odense (DK); Oliver Schoor, Tubingen (DE)

(73) Assignee: IMMATICS BIOTECHNOLOGIES GMBH, Tuebingen (DE)

(21) Appl. No.: 12/915,930

(22) Filed: Oct. 29, 2010

Related U.S. Application Data
(62) Division of application No. 10/549,718, filed on Aug. 17, 2007.

(30) Foreign Application Priority Data
Mar. 24, 2003 (DE) 103 13 819.6

(51) Int. Cl. A61K 38/10; C07K 7/06; C07K 7/08; A61K 38/08; A61P 35/00; A61K 39/00; C07K 16/00; A61K 38/00; C07H 21/04; A61K 31/7088; C12N 15/63; C12N 5/078; C12N 5/0783; C12N 5/10; G01N 33/53

(52) U.S. Cl. 424/185.1; 530/328; 530/327; 530/329; 514/19.2; 514/19.3; 514/19.4; 530/387.1; 514/1.1; 536/23.1; 514/44 R; 435/320.1; 435/325; 435/7.24

(57) ABSTRACT

The invention relates to a tumour-associated peptide with an amino acid sequence that is selected from the group consisting of SEQ ID NO: 1 to SEQ ID NO: 101 of the attached sequence protocol, wherein the peptide has the ability to bind to a molecule of the human major-histocompatibility-complex (MHC) class-I. In addition, the invention relates to the use of the peptides and the nucleic acids encoding for the peptides for the production of a medicament, and for the treatment of tumorous diseases and/or adenomatous diseases. Furthermore, a pharmaceutical composition is described that has at least one of the peptides.

### RELATED APPLICATIONS

This application is divisional of U.S. patent application Ser. No. 10/549,718, submitted to the United States Patent and Trademark Office on Sep. 16, 2005 and granted a filing date of Aug. 17, 2007 under 35 U.S.C. §371(c)(1), (c)(2), and (c)(4), which is a National Stage Application of International Application Number PCT/EP04/03077, filed Mar. 23, 2004, which claims priority to German Patent Application Number 103 13 819.6, filed Mar. 24, 2003, the contents of which are hereby incorporated by reference in their entirety.

### BACKGROUND

The present invention relates to tumour-associated peptides that are able to bind to a molecule of the human major-histocompatibility-complex (MHC), class I.

Such peptides are used, for example, in the immunotherapy of tumorous diseases.

The recognition of tumour-associated antigens (TAA) by components of the immune system plays a prominent role in the elimination of tumour cells by the immune system. This mechanism is based on the prerequisite that qualitative or quantitative differences exist between tumour cells and normal cells. In order to effect an anti-tumour-response, the tumour cells have to express antigens against which an immunological response takes place that is sufficient for the elimination of the tumour.

Involved in the rejection of tumours are in particular CD8-expressing cytotoxic T-lymphocytes (in the following CTLs). For triggering of such an immune reaction by cytotoxic T-cells, foreign proteins/peptides have to be presented to the T-cells. T-cells recognise antigens as peptide fragments only, if these are presented by MHC-molecules on cellular surfaces. These MHC-molecules (“major histocompatibility complex”) are peptide receptors that normally bind peptides within the cell in order to transport them to the cellular surface. This complex of peptide and MHC-molecule can be recognised by the T-cells. The MHC-molecules of the human are also designated as human leukocyte-antigens (HLA).

There are two classes of MHC-molecules: MHC-class-1-molecules, that are found on most of the cells with a nucleus, present peptides that are generated by proteolytic degradation of endogenous proteins. MHC-class-II-molecules are only present on professional antigen-presenting cells (APCs), and present peptides of exogenous proteins that are taken up and processed by APCs during the course of endocytosis. Complexes of peptide and MHC-class-I are recognised by CD8-positive cytotoxic T-lymphocytes, complexes of peptide and MHC-class-II are recognised by CD4-helper-T-cells.

In order for a peptide to trigger a cellular immune response, it must bind to an MHC-molecule This process is dependent from the allele of the MHC-molecule and the amino acid sequence of the peptides. MHC-class-1-binding peptides are usually 8-10 residues in length, and contain two conserved residues (“anchors”) in their sequence that interact with the corresponding binding groove of the MHC-molecule.

In order for the immune system to be able to start an effective CTL-response against tumour-derived peptides, these peptides must not only be able to bind to the particular MHC-class-I-molecules that are expressed by the tumour cells, but they must also be recognised by T-cells having specific T-cell receptors (TCR).

The main goal for the development of a tumour vaccine is the identification and characterisation of tumour-associated antigens that are recognised by CD8+ CTLs.

The antigens that are recognised by the tumour-specific cytotoxic T-lymphocytes or their epitopes, respectively, can be molecules from all classes of proteins, such as, for example, enzymes, receptors, transcription factors, etc. Another important class of tumour associated antigens are tissue-specific structures, such as, for example, CT (“cancer testis”)-antigens that are expressed in different kinds of tumours, and in healthy tissue of testes.

In order for the proteins to be recognised by the cytotoxic T-lymphocytes as tumour-specific antigen, and in order to be able to be used in a therapy, particular prerequisites must be present: The antigen shall mainly be expressed by tumour cells, not by normal tissues or only in lower amounts than in the tumours. It is furthermore desirable that the respective antigen is present not only in one kind of tumour, but also in high concentration in others. In addition, absolutely essential is the presence of epitopes in the amino acid sequence of the antigens, since those of a tumour-associated antigen-derived peptide (“immunogenic peptides”) shall lead to a T-cell-response, whether in vitro or in vivo.

Therefore, TAAs provide a starting point for the development of a tumour vaccine. The methods for the identification and characterisation of the TAAs, on the one hand, are based on the use of CTLs that are already induced in patients, or are based on the generation of differential transcription profiles between tumour and normal tissues.

The identification of genes that are overexpressed in tumour tissues, or that are selectively expressed in those tissues, nevertheless, did not deliver precise information for a use of the antigens that are transcribed by these genes in immunotherapy. This is due to the fact that in each case only single epitopes of these antigens are suitable for such a use, since only the epitopes of the antigens—and not the whole antigen—trigger a T-cell-response through MHC-presentation. It is therefore important to select those peptides of overexpressed or selectively expressed proteins that are presented with MHC-molecules, whereby starting points for the specific tumour-recognition by cytotoxic T-lymphocytes can be obtained.

### DETAILED DESCRIPTION OF THE INVENTION

In view of this background, it is an object of the present invention to provide at least one novel amino acid sequence for such a peptide that has the ability to bind to a molecule of the human major-histocompatibility-complex (MHC) class-I.

According to the invention, this object is solved by the provision of a tumour-associated peptide with an amino acid sequence that is selected from the group consisting of SEQ ID NO: 1 to SEQ ID NO: 101 of the attached sequence protocol, wherein the peptide has the ability to bind to a molecule of the human major-histocompatibility-complex (MHC) class-I.

Thereby, the object that forms the basis of the invention is completely solved.

It shall be understood that the peptides from the tumour as identified can be synthesised or brought to expression in cells in order to obtain larger amounts thereof, and for the use for the purposes as mentioned below.

The inventors could isolate and identify the above-mentioned peptides as specific ligands of MHC-class-1-molecules from tumour tissues. Thereby, the term “tumour-associated” designates peptides that were isolated and identified from tumour material. These peptides, that are presented on real (primary) tumours therefore underlie antigen processing in a tumour-cell.

The specific ligands can be used in cancer therapy, e.g. in order to induce an immune response against tumour cells that express the corresponding antigens from which the peptides are derived.

On the one hand, such an immune response can be achieved in vivo in the form of an induction of CTLs. For this, the peptide, for example in the form of a pharmaceutical composition, is administered to a patient who suffers from a tumorous disease that is associated with the TAA.

On the other hand, a CTL-response towards a tumour that expresses the antigens from which the peptides are derived can also be triggered ex vivo. For this, the CTL-precursor cells are incubated together with antigen-presenting cells, and the peptides. Subsequently, the thus stimulated CTL are cultured, and these activated CTL are administered to the patient.

Furthermore, the possibility exists to load APC ex vivo with the peptides, and to administer these loaded APCs to the patient who expresses the antigen in the tumorous tissue, from which the peptide is derived from. The APCs, in turn, then are able to present the peptide to the CTLs in vivo, and activate these.

Nevertheless, the peptides according to the invention can be used as diagnostic reagents.

Thus, using the peptides it can be identified, whether CTLs are present in a CTL-population that are specifically directed against a peptide, or are induced by a therapy.

In addition, the increase of precursor T-cells can be tested for with the peptides that exhibit a reactivity against the defined peptide.

Furthermore, the peptide can be used as a marker in order to monitor the progression of a disease of a tumours that expresses the antigen from which the peptide is derived.

In the attached table 1, the identified peptides are listed. Furthermore, in said table the proteins are given from which the peptides are derived, and the respective positions of the peptides in the respective proteins. Thereby, the English designations of the proteins were maintained in order to avoid mistakable translations. Furthermore, the Acc-numbers are given, respectively that are maintained in the Genbank of the “National Centre for Biotechnology Information” of the National Institute of Health (see http: www.ncbi.nlm.nih.gov).

The inventors could isolate the peptides (or ligands) from renal cell tumours of two patients, RCC68, and RCC44.

From the tumours of the patients, 101 ligands could be identified, that were bound to the HLA-subtypes HLA-A*02, HLA-A*29, HLA-B*15 or HLA-B*45 (patient RCC68) and to HLA-A*3201, HLA-A*1101, HLA-B*4002, HLA-B*2705 or HLA-Cw*0202 (patient RCC44).

Some of the ligands were derived from strongly expressed so-called “housekeeping” genes that are uniformly expressed in most tissues, nevertheless, many were characterised by tissue specific and tumour specific expression.

Thus, some peptides could be identified that are derived from proteins that are overexpressed, particularly in tumorous tissue. Thus, for example, fragments of vimentin (ALRDVRQQY, position 268-276, SEQ ID NO: 7; EENFAVEA, position 348-355, SEQ ID NO: 15; MEENFAVEA, position 347-355, SEQ ID NO: 45; NYIDKVRFL, position 116-124, SEQ ID NO: 50) could be identified. Young et al., expression profiling of renal epithelial neoplasms: a method for tumor classification and discovery of diagnostic molecular markers, 2001, Am. J. Pathol., 158:1639-1651) showed that this protein was overexpressed in tissue of renal cell tumours.

In addition, the inventors could identify, amongst others, ligands that are derived from alpha-catenin, (LQHPDVAAY, position 229-237, SEQ ID NO: 43), and beta-catenin (AQNAVRLHY, position 481-489, SEQ ID NO: 8).

Furthermore, the inventors could show in own experiments that by using of exemplary selected peptides it was possible to generate cytotoxic T-lymphocytes (CTLs) in vitro that were each specific for the selected peptides. Using these CTLs, tumour cells could selectively be killed which expressed the corresponding proteins, and which, in addition, were derived from different tumour cell lines of different patients. Furthermore, said CTLs, for example, also lysed dendritic cells that were “pulsed” (loaded) in advance with the respective peptides. Thus, it could be shown that, with the peptides according to the present invention as epitopes, human T-cells in vitro could be activated in vitro. Accordingly, the inventors could not only show that CTLs that were obtained from peripheral blood-mononuclear-cells (PBMNCs) of a patient, and which were specific for a particular peptide, could kill cells of the same kind of tumour of another patient. In addition, the inventors showed that also cells of other kinds of tumours could be lysed with these CTLs.

In a preferred embodiment also peptides could be used for a stimulation of an immune response that exhibited the SEQ ID NO: 1 to 101, and wherein at least one amino acid is replaced by another amino acid having similar chemical properties.

With respect to the respective MHC-subtypes, these are, for example, the anchoring amino acids, which can be replaced by amino acids with similar chemical properties. Thus, for example, in case of peptides which are associated with the MHC-subtype HLA-A*02 leucine at position 2 can be replaced by isoleucine, valine or methionine, and vice versa, and at the C-terminus leucine by valine, isoleucine, and alanine, that all have non-polar side chains.

It is furthermore possible, to use peptides with the SEQ ID NO: 1 to 101, that N- or/and C-terminally exhibit at least one additional amino acid, or wherein at least one amino acid is deleted.

Furthermore, peptides with the SEQ ID NO: 1 to 101 can be used, wherein at least one that amino acid is chemically modified.

Thereby, the varying amino acid(s) is(are) chosen in such a manner that the immunogenicity of the peptide is not affected by the variation, i.e. it has a similar binding affinity to the MHC-molecule and the ability for a T-cell-stimulation.

According to the invention, the peptide can be used for the treatment of tumorous diseases and/or adenomatous diseases.

Thereby, the tumorous diseases to be treated comprise, for example, renal, breast, pancreatic, stomach, testes, and/or skin cancer. In doing so, the listing of the tumorous diseases is only exemplary, and shall not limit the scope of use. The fact that the peptides according to the invention are suitable for such use, could be demonstrated by the inventors in their own experiments. Therein, it was shown that specifically generated CTL that were specific for particular peptides could effectively and selectively kill tumour cells.

In general, several application forms are possible for a use of tumour-associated antigens in a tumour vaccine. Tighe et al., 1998, Gene vaccination: plasmid DNA is more than just a blueprint, Immunol. Today 19(2):89-97, described that the antigen can be administered either as recombinant protein together with suitable adjuvants or carrier systems, or as the cDNA encoding for the antigen in plasmid vectors. In these cases, in order to evoke an immune response, the antigen must be processed and presented in the body of the patient by antigen-presenting cells (APCs).

Melief et al., 1996, peptides-based cancer vaccines, Curr. Opin. Immunol. 8:651-657, showed an additional possibility, namely the use of synthetic peptides as vaccine.

For this, in a preferred embodiment, the peptide can be used with the addition of adjuvants, or else in singular form.

The granulocyte-macrophage-colony-stimulating-factor (GM-CSF) can, for example, be used as adjuvant. Further examples for such adjuvants are aluminium hydroxide, emulsions of mineral oils, such as, for example, Freund's adjuvant, saponines or silicon compounds.

The use together with an adjuvant offers the advantage that the immune response that is triggered by the peptide can be enhanced and/or that the peptide is stabilised.

In another preferred embodiment, the peptide is used bound to an antigen-presenting cell.

These measure has the advantage that the peptides can be presented to the immune system, in particular the cytotoxic T-lymphocytes (CTLs). In doing so, the CTLs can recognise the tumour cells, and specifically kill them. As antigen-presenting cells, for example, dendritic cells, monocytes or B-lymphocytes are suitable for such a use.

Thereby, the cells can be loaded, for example ex vivo, with the peptides. On the other hand, the possibility exists to transfect the cells with the DNA encoding for the peptides or the corresponding RNA in order to then bring the peptides to an expression on the cells.

The inventors could show in own experiments that it is possible to specifically load dendritic cells (DC) with specific peptides, and that these loaded dendritic cells activate peptide-specific CTLs. This means, that the immune system can be stimulated in order to develop CTLs against the tumours expressing the corresponding peptides.

Thereby, the peptide-carrying antigen-presenting cells can either be used directly, or activated before a use with, for example, the heat shock-protein gp96. This heat shock protein induces the expression of MHC-class I-molecules, and of costimulating molecules, such as B7, and additionally stimulates the production of cytokines. Thereby, the overall triggering of an immune response is promoted.

In another preferred embodiment, the peptides are used for the labelling of leukocytes, in particular of T-lymphocytes.

This use is of advantage if, using the peptides, it shall be elucidated, if CTLs that are specifically directed against a peptide are present in a CTL-population.

Furthermore, the peptide can be used as a marker for judging the progression of a therapy in a tumorous disease.

The peptide can be used also in other immunisations or therapies for the monitoring of the therapy. Thus, the peptide can not only be used therapeutically, but also diagnostically.

In another embodiment, the peptides are used for the production of an antibody.

Polyclonal antibodies can be obtained in a common manner by immunisation of animals by means of injection of the peptides, and subsequent purification of the immunoglobulin.

Monoclonal antibodies can be produced following standard protocols, such as, for example, described in Methods Enzymol. (1986), 121, Hybridoma technology and monoclonal antibodies.

In another aspect, the invention furthermore relates to a pharmaceutical composition that contains one or several of the peptides.

This composition, for example, is used for parenteral administration, for example, subcutaneous, intradermal or intramuscular or oral administration. For this, the peptides are dissolved or suspended in a pharmaceutically acceptable, preferably aqueous, carrier. In addition, the composition can contain auxiliary agents, such as, for example, buffers, binding agents, diluents, etc.

The peptides can also be administered together with immune stimulating substances, e.g. cytokines A comprehensive demonstration of auxiliary agents that can be used in such a composition, is, for example, shown in A. Kibbe, Handbook of Pharmaceutical Excipients, 3. Ed., 2000, American Pharmaceutical Association and pharmaceutical press.

Thereby, the agent can be used for the prevention, prophylaxis and/or therapy of tumorous diseases and/or adenomatous diseases.

The pharmaceutical agent, that at least contains one of the peptides with the SEQ ID NO: 1 to 101, is administered to a patient that suffers from a tumorous disease which is associated with the respective peptide or antigen. By this, a tumour-specific immune response on the basis of tumour-specific CTLs can be triggered.

Thereby, the amount of the peptide or the peptides as present in the pharmaceutical composition is a therapeutically effective amount. Thereby, the peptides as contained in the composition can also bind to at least two different HLA-types.

In another aspect, the present invention relates to nucleic acid molecules that encode for the peptides having the SEQ ID NO: 1 to 101, as well as the use of at least one of the nucleic acid molecules for producing a medicament for the therapy of tumorous diseases and/or adenomatous diseases.

Thereby, the nucleic acid molecules can be DNA- or RNA-molecules, and also be used for the immunotherapy of cancerous diseases. In doing so, the peptide that is induced by the nucleic acid molecule induces an immune response against tumour cells that express the peptide.

According to the invention, the nucleic acid molecules can also be present in a vector.

In addition, the invention relates to cells which have been genetically modified with the aid of the nucleic acid molecule that encodes for the peptides in such a manner that the cell produces a peptide with the SEQ ID NO: 1 to 101.

For this, the cells are transfected with the DNA encoding for the peptides or the corresponding RNA, whereby the peptides are brought to an expression on the cells. For such a use as antigen-presenting cells, for example, dendritic cells, monocytes or other human cells are suited, that express suitable molecules for the co-stimulation, such as, for example, B7.1 or B7.2.

The invention further relates to a diagnostic method, wherein the presence of one of the novel peptides is used as a diagnostic marker, as well as to a method for the treatment of a pathological condition, wherein an immune response against a protein of interest is triggered, wherein a therapeutically effective amount of at least one of the novel peptides is administered.

The inventors have realised that the novel peptides can also be used as markers for a pathological condition, such that a respective diagnostic method, wherein a blood sample of the patient is taken and is examined in a common manner for the presence of lymphocytes that are directed against one of the novel peptides, can be used as an early diagnosis or for the targeted selection of a suitable treatment.

Furthermore, the invention relates to an electronic storage medium, which contains the amino acid sequence of at least one of the novel peptides and/or the nucleic acid sequence of nucleic acid molecules that encode for the novel peptides.

Starting from this storage medium, then, in case of the presence of a corresponding indication, the information for the peptides that are suitable for the treatment of the pathological condition can be provided quickly.

It shall be understood that the above mentioned features and the features to be explained in the following can not only be used in the respectively given combination, but also in a unique positioning without departing from the scope of the present invention.

Embodiments of the invention are explained in the following examples.

### EXAMPLES

### Example 1

### 1.1. Patient Samples

Two samples were obtained from the department for urology, Universität Tübingen, that were derived from patients that suffered from histologically confirmed renal cell tumours. Both patients had received no pre-surgical therapy. Patient No. 1 (in the following designated RCC68) had the following HLA-typing: HLA-A*02 A*29 B*15 B*45; patient No. 2 (in the following designated RCC44) HLA-A*3201 A*1101 B*4002 B*2705 Cw*0202,

### 1.2. Isolation of the MHC-Class-I-Bound Peptides

The shock-frozen tumour samples were processed as already described in Schirle, M. et al., Identification of tumor-associated MHC class I ligands by a novel T cell-independent approach, 2000; European Journal of Immunology, 30:2216-2225. The peptides were isolated according to standard protocols, and in particular by using the monoclonal antibody W6/32 that is speCific for HLA-class-I-molecules, or the monoclonal antibody BB7.2 that is specific for HLA-A2. Barnstable, C. J. et al., Production of monoclonal antibodies to group A erythrocytes, HLA and other human cell surface antigens-new tools for genetic analysis, 1978, Cell, 14:9-20 and Parham, P. & Brodsky, F. M., Partial purification and some properties of BB7.2. A cytotoxic monoclonal antibody with specificity for HLA-A2 and a variant of HLA-A28, 1981, Hum. Immunol., 3:277-299, describe the production and uses of these antibodies.

### 1.3. Mass Spectroscopy

The peptides were separated by “reversed phase HPLC” (SMART-system, mRPC C2/C18 SC 2.1/19, Amersham Pharmacia Biotech), and the fractions as obtained were analysed by nano-ESI MS. This was done as described in Schirle, M. et al., Identification of tumorassociated MHC class I ligands by a novel T cell-independent approach, 2000, European Journal of Immunology, 30:2216-2225.

The peptides that were obtained from tumorous tissue were identified by capillary-LC-MS as just mentioned, nevertheless with slight changes: 100 pl of each of the samples were loaded, desalted, and pre-concentrated on a 300 pm*5 mm C18 p-pre-column (LC Packings). The solvent and the sample were added by means of a syringe pump (PHD 2000, Harvard apparatus, Inc.) with a sealed 100 pl-syringe (1710 RNR, Hamilton) with a speed of 2 pl/min. For the separation of the peptides, the pre-concentration-column was disposed before a 75 tilla*250 mm C-18-column (LC Packings). Subsequently, a binary gradient with 25-60% B was run within 70 min, whereby the flow rate was reduced from 12 pl/min to about 300 nl/min, and in particular by using a TEE-connection (ZT1C, Valco), and a 300 p.m*150 mm C-18-column.

In order to ensure that the system was free of residual peptides, in each case a blank sample was measured. Online-fragmentation was performed as described, and the spectra of the fragments were analysed manually. The database searches (NCBInr, EST) were performed using MASCOT (http://www.matrixscience.com).

1.4. Identification of the MHC-Class-1-Ligands from Tumorous Tissue of the Patients RCC68 and RCC44

In the attached sequence protocol and in the attached table 1 the ligands are listed that were bound to the HLA-molecules of the patients RCC68 and TCC44. The peptides that were associated with HLA-A*02 exhibited the allele-specific peptide motif: Thus, at position 2 leucine, valine, isoleucine, alanine or methionine, and at the C-terminus leucine, valine, isoleucine, or alanine could be found. Most of the ligands were derived from so-called “housekeeping”-proteins, nevertheless, also ligands from proteins could be identified which are associated with tumours. Thus, for example, fragments of vimentin (ALRDVRQQY, position 268-276, SEQ ID NO: 7; EENFAVEA, position 348-355, SEQ ID NO: 15; MEENFAVEA, position 347-355, SEQ ID NO: 45; NYIDKVRFL, position 116-124, SEQ ID NO: 50) could be identified. Young et al. (Expression profiling of renal epithelial neoplasms: a method for tumor classification and discovery of diagnostic molecular markers, 2001, Am. J. Pathol., 158:1639-1651) showed that this protein was overexpressed in tissue of renal cell tumours.

### 1.5. Detection of Peptide-Specific T-Cells in the Normal CD8+-T-Cell-Repertoir

For a detection of peptide-specific T-cells, mononuclear cells from peripheral blood of healthy patients were stained with the respective HLA-A*subtype-tetramers that were constituted with the respective peptides: For a production of the tetramers, recombinant HLA-A*subtype-molecules were constituted with the peptides in vitro, purified by gel filtration, biotinylated, and mixed with streptavidin for a linking of the monomers.

In general, the results of the double stainings were evaluated by analysis using of FACS, and the specific binding of the peptide-tetramers was detected.

### Example 2

In order to analyse the presentation of the selected peptides by tumour cells, and the recognition of the peptides by CTLs to, CTLs that were specific for the selected peptides were induced in vitro. For this, dendritic cells (DCs) were used that were derived from peripheral blood-mononuclear-cells (PBMNCs) of healthy donors, that had the same respective HLA-(sub)type.

### 2.1. Obtaining of DCs

The DCs were isolated by Ficoll/Paque-(Biochrom, Berlin, Germany)-density gradient-centrifugation of PBMNCs from heparinised blood. The heparinised blood was obtained from “buffy coat”-preparations of healthy donors of the blood bank of the Universität Tübingen. The cells were seeded on 6-well-plates (Falcon, Heidelberg, Germany) (1×10⁷ cells/3 ml per well) in RP10 medium (RPMI 1640, supplemented with 10% heat-inactivated foetal calf serum and with antibiotics). Following a 2-hour incubation at 37° C. and 5% CO₂, the non-adhering cells were removed, and the adhering blood monocytes were cultivated in RP10 medium, whereby the following cytokines were added into the medium as supplement: human recombinant GM-CSF (granulocyte macrophage colony stimulating factor; Leukomax, Novartis; 100 ng/ml), interleukin IL-4 (R&D Systems, Wiesbaden, Germany; 1000 IU (ml), and TNF-α (Tumor-Nekrose-Faktor a) (R&D Systems, Wiesbaden, Germany; 10 ng/ml).

### 2.2. Synthesis of the Peptides

The exemplary selected peptides were synthesised on a peptide-synthesiser (432A, Applied Biosystems, Weiterstadt, Germany) using F-moc (9-fluoroenylmethyloxycarbonyl)—protective groups, and analysed by “reversed phase” HPLC and mass spectroscopy. By this way, sufficient amounts of the identified peptides could be produced.

### 2.3. Induction of an Antigen-Specific CTL-Response Using Restringed Synthetic Peptides

For an induction of CTLs, the DCs (5×10⁵) as obtained in step 2.1. were pulsed for 2 hours with 50 μg/ml of the peptides obtained from step 2.2., subsequently washed and incubated with 2.5×10⁶ autologous PBMC in RP10 medium_{—} After a 7-day cultivation period, the cells were restimulated with autologous, peptide-pulsed PBMNCs. In doing so, 1 ng/ml human recombinant interleukin 1L-2 (R&D Systems) was added on day 1, 3, and 5. The cytotoxic activity of CTLs that were induced by this way was examined on day 5 following the last restimulation by means of a standardised ⁵¹Cr-release-assay (see below at 2.4.: CTL-assay).

### 2.4. CTL-Assay

For the CTL-assays, tumour cells, peptide-pulsed cells of different cell lines, and autologous DCs were used as target-cells. Peptide-pulsed cells were pulsed with 50 μg/ml peptide for 2 hours. All target cells were (⁵¹Cr) labelled in RP10 medium (RPMI 1640, supplemented with 10% heat-inactivated foetal calf serum and with antibiotics) for 1 hour at 37° C. with [⁵¹Cr] sodium chromate. Subsequently, 10⁴ cells/per each well were given on a 96-well-plate with rounded bottoms. Different amounts of CTLs were added in order to reach a final volume of 200 μl, with subsequent incubation for 4 hours at 37° C. Thereafter, the supernatants (50 μl/well) were harvested and counted in a beta-plate-counter. The specific lysis was calculated in percent as follows: 100×(experimental release−spontaneous release/maximal release−spontaneous release). The spontaneous and the maximal release were each determined in the presence of either medium or 2% triton X-100.

### 2.5. Results of the CTL-Induction

### a) CTL-Cytotoxic Activity Versus Peptide-Pulsed DCs

In ⁵¹Cr-release-assays (see at 2.4.) the cytotoxic activity of induced CTLs (see at 2.3.) versus T2- or DC-cells was tested. The T2-cell line is HLA-A*02-positive and TAP (transporter associated with antigen processing)—deficient; (TAP-peptide-transporters transport peptide-fragments of a protein antigen from the cytosol into the endoplasmatic reticulum, where they associate with MHC-molecules).

The results of these release-assays show that with CTL-cell lines that were obtained after 2-week restimulation, an antigen-specific killing of the cells could be achieved: Only those cells were killed by an increasing amount of CTL that presented each of the selected peptides; the control cells that were loaded with irrelevant peptides were not killed. Thereby, the specificity of the cytolytic activity could be shown.

### b) CTL-Cytotoxic Activity Versus Tumour Cell Lines

In a next step, it was tested again by a ⁵¹Cr-release-assay, whether the CTLs that were specific for the selected peptides recognise and lyse tumour cells that endogenously express the selected peptides.

For this, different ⁵¹Cr-labelled cell lines expressing the corresponding HLA-molecules were used: HCT 116 (colon cancer; obtained from Prof G. Pawelec, Tübingen, Germany), A 498, MZ 1257 and MZ 1774 (renal cell carcinoma; obtained from Prof A. Knuth, Frankfurt, Germany), MCF-7 (breast cancer; commercially obtained from the ATCC, American Type Culture Collection), Mel 1479 (melanoma; obtained from Prof. G. Pawelec, Tübingen, Germany), and U 266 (multiple myeloma; obtained from Prof G. Pawelec, Tübingen, Germany). These cell lines express particular proteins as target structures (“targets”).

The B-cell line Croft (EBV (Epstein-Barr-Virus)-immortalised; HLA-A*02-positive; obtained from O. J. Finn, Pittsburgh, USA) and the cell line SK-OV-3 (ovarian tumour; HLA-A*03-positive; obtained from O. J. Finn, Pittsburgh, USA) were included in the study as negative controls. K 562 cells (obtainable, for example, at the Deutschen Sammlung von Mikroorganismen and Zellkulturen, DSMZ; ACC 10) were used in order to determine the activity of natural killer cells (NK), since this cell line is highly sensitive against these killer cells.

All cell lines were cultivated in RP10 medium (RPMI 1640, supplemented with 10% heat-inactivated foetal calf serum and with antibiotics).

With the above tumour cell lines and the CTLs as induced at 2.3., ⁵¹Cr-release assays (see at 2.4.) were performed.

In these tests, the CTLs that were each specific for the selected peptides efficiently lysed tumour cells that expressed both the corresponding HLA-molecule as well as the selected peptides. The specific lysis was—as given above at 2.4.—measured by the ⁵¹Cr-release. In contrast, the control cell line SK-OV-3 (HLA-A-*02-negative) was not lysed by the CTLs that were induced by the peptides that were bound by HLA-A*02. This showed that the peptides must be presented in connection with the corresponding HLA-molecules on the tumour cells in order to efficiently lyse the target-cells. Furthermore, by this the antigen-specificity and the MHC-restriction of the CTLs is confirmed.

In addition, the CTL-cells that were induced in vitro by the peptides did not recognise the cell line K562, demonstrating that the cytotoxic activity was not mediated by natural killer cells (NK)-cells.

### c) Inhibition-Assays

In order to further verify the antigen-specificity and the MHC-restriction of the in-vitro induced CTLs, inhibitions-assays were performed with non-⁵¹Cr-labelled (“cold”) inhibitor-cell lines.

Here, the ability of peptide-pulsed cell lines was analysed to inhibit the lysis of tumour cells, or to be competitive. For this, an excess of inhibitor (i.e. of pulsed, non-labelled cells) was used. The ratio of the inhibitor (peptide-pulsed cells) to target (tumour cells) was 20:1. Upon lysis of the inhibitor-cell lines, no ⁵¹Cr could be released since the inhibitor-cell lines were non-labelled.

The cell line T2 (HLA-A*02; TAP—deficient; see at 2.5.a)) was used as inhibitor. T his cell line T2 was pulsed before the assays with each of the relevant peptides, or an irrelevant control peptide.

In the absence of the inhibitor-cells, a lysis of the tumour cells by CTL was observed. It could furthermore be shown that, in case of an excess of inhibitor-target, no lysis of the tumour cells took place (and thus no ⁵¹Cr-release), as long as the inhibitor-target was pulsed with the corresponding peptides. The activity of the CTLs was directed to the non-labelled T2-cells present in excess, such that these and not the tumour cells were lysed. The T2-cells that were pulsed with an irrelevant peptide could not inhibit the lysis of the tumour cells by the CTLs, such that released ⁵¹Cr could be measured.

The MHC-restriction and the antigen-specificity of the cytotoxic activity that was mediated by the HLA-A*02-peptide-induced CTL could be confirmed using a HLA-A*02-specific monoclonal antibody, and in an inhibition-assay with non-labelled (“cold”) inhibitor: The A 498-tumor cells were blocked by the addition of the HLA-A*02-specific antibody (monoclonal antibody BB7.2, IgG2b, obtained from S. Stefanovic, Tubingen), such that they were not lysed by the addition of the CTLs, and no ⁵¹Cr was released. An unspecific antibody served as control that did not block HLA-A*02-molecules (ChromPure mouse IgG, Dianova, Germany). For these inhibition-experiments, the cells were incubated 30 min. with 10 μg/ml antibody before seeding on the 96-well-plates.

It could furthermore be found that the T2-competition-cell line that was pulsed with an irrelevant peptide could not inhibit the CTL-mediated lysis of the tumour cell line A 498, but that the T2-inhibitor-cell line pulsed with the corresponding peptide could inhibit the lysis of the tumour-cell line, such that in the latter case no ⁵¹Cr-release could be measured.

### d) Specific Lysis of Transfected DCs

In a next experiment, the cytotoxic activity of the CTLs was analysed in an autologous experimental setting. For this, autologous DCs that were obtained from the same PBMNCs as those that were used for the CTL-induction (see at 2.2.) were used as target cells. Before performing the CTL-assay, the DCs were electroporated with RNA that was isolated earlier either from tumour-cell lines, or that represented control-RNA. The total-RNA was isolated from the tumour cells using the QIAGEN Rneasy mini kit (QIAGEN, Hilden, Germany) in accordance with the manufacturers instructions. Amount and purity of the RNA was determined photometrically, and stored in aliquots at −80° C.

Before the electroporation on day 6, immature DCs were washed two times with serumfree X-VIVO 20 medium (BioWhittaker, Walkersville, USA), and resuspended in a final concentration of 2×10⁷ cells/ml. Subsequently, 200 μl of the cell suspension were mixed with 10 μg of the total-RNA, and electroporated in a 4 mm cuvette by means of an Easyject™ (Peqlab, Erlangen, Germany) (parameters: 300 V, 150 μF 1540Ω, pulse time: 231 ms). Following the electroporation, the cells were immediately transferred into RP10 medium and again given into the incubator. More than 80% of the cells were viable following the electroporation.

After performing the CTL-assays with CTLs that were induced by the selected peptides (see at 2.4.), a specific lysis of DCs could be observed which were electroporated with RNA of peptide-expressing tumour-cell lines. In contrast, DCs that were electroporated with RNA of a non-peptide-expressing tumour-cell line, were not lysed.

This shows that—following transfection of the DCs with RNA of peptide-positive tumour-cells—the identified peptides are processed and presented.

e) Induction of Peptide-Specific CTLs in a Patient with Chronic Lymphatic Leukaemia

In an additional experiment, CTLs that were specific for selected peptides were generated from PBMNCs of a patient with chronic lymphatic leukaemia (CLL). Furthermore, the autologous primary CLL-cells and DCs of this patient were used as ⁵¹Cr-labelled targets in an assay, wherein a ⁵¹Cr-release was mediated by the peptide-induced CTLs. As a result, both the autologous DCs of this patient that were pulsed with the selected peptides, as well as the autologous CLL-cells were lysed by the peptide-induced CTLs. In contrast, DCs that were pulsed with an irrelevant peptide were not lysed. In addition, non-malignant B-cells and the cell line K 562 were not lysed by the CTLs.

The specificity of the CTL-response was confirmed in a target-inhibition-assay, whereby the cell line T2 (see above) was used as inhibitor-cells which were pulsed with each of the selected peptides or with an irrelevant peptide. Also in this case, the CTLs that were induced by using the peptides lysed the inhibitor-cell lines present in excess that were pulsed with the relevant peptides, such that in this case the ⁵¹Cr-labelled tumour cells were not lysed.

In summary, therefore the inventors could show that the peptides as identified represent promising substances in the context of an immunotherapy in a multitude of (tumorous-) diseases.

TABLE 1

| | sequence | Position/Gene type | Acc. No. | SEQ ID-No. |
|---|---|---|---|---|
| 1. | AAFPGASLY | 63-7 | NM_014764 | SEQ ID-No. 1 |
| | | DAZ associated protein 2 | | |
| 2. | AELATRALP | 137-145 | NM_002230 | SEQ ID-No. 2 |
| | | junction placoglobin | | |
| 3. | AFFAERLYY | 397-405 | NM_001156 | SEQ ID-No. 3 |
| | | annexin A7 | | |
| 4. | ALATLIHQV | 26-34 | NM_016319 | SEQ ID-No. 4 |
| | | COP9 constitutive | | |
| | | photomorphogenic homolog | | |
| | | subunit 7A (Arabidopsis) | | |
| 5. | ALAVIITSY | 318-326 | NM_005765 | SEQ ID-No. 5 |
| | | ATPase, H+ transporting, | | |
| | | lysosomal (vacuolar proton pump) | | |
| | | membrane sector associated protein | | |
| | | M8-9 | | |
| 6. | ALQEMVHQV | 806-814 | NM_006403 | SEQ ID-No. 6 |
| | | enhancer of filamentation 1 | | |
| 7. | ALRDVRQQY | 268-276 | NM_003380 | SEQ ID-No. 7 |
| | | vimentin | | |
| 8. | AQNAVRLHY | 481-489 | NM_001904 | SEQ ID-No. 8 |
| | | catenin (cadherin-associated | | |
| | | protein), beta 1, 88 kDa | | |
| 9. | AQPGFFDRF | 1006-1014 | NM_001849 | SEQ ID-No. 9 |
| | | collagen, type VI, alpha 2 | | |
| | | (COL6A2), transcript variant 2C2 | | |
| 10. | AVCEVALDY | 2260-2268 | NM_003128 | SEQ ID-No. 10 |
| | | spectrin, beta, non-erythrocytic 1 | | |
| 11. | AVLGAVVAV | 161-169 | M12679 | SEQ ID-No. 11 |
| | | Cw1 antigen | | |
| 12. | DAILEELSA | 154-162 | NM_024591 | SEQ ID-No. 12 |
| | | hypothetical protein FLJ11749 | | |
| 13. | EEHPTLLTEA | 101-110 | NM_002388 | SEQ ID-No. 13 |
| | | actin, alpha 2, smooth muscle, aorta | | |
| 14. | EEMPQVHTP | 715-723 | NM_002388 | SEQ ID-No. 14 |
| | | MCM3 minichromosome | | |
| | | maintenance deficient 3 (S. cerevisiae) | | |
| 15. | EENFAVEA | 348-355 | NM_00380 | SEQ ID-No. 15 |
| | | vimentin | | |
| 16. | EENKLIYTP | 56-64 | NM_012106 | SEQ ID-No. 16 |
| | | binder of Arl Two | | |
| 17. | FAEGFRAL | 110-118 | NM-018834 | SEQ ID-No. 17 |
| | | v-jun sarcoma virus 17 oncogen | | |
| | | homolog (avian | | |
| 18. | FFGETSHNY | 235-243 | NM_108834 | SEQ ID-No. 18 |
| | | matrin 3 | | |
| 19. | FLPHMAYTY | 931-939 | NM_014795 | SEQ ID-No. 19 |
| | | zinc finger homeobox 1 b | | |
| 20. | GEPRFISVGY | 42-51 | Z46810 | SEQ ID-No. 20 |
| | | major histocompatibility complex, | | |
| | | class 1, C | | |
| 21. | GLATDVQTV | 55-63 | NM_002795 | SEQ ID-No. 21 |
| | | proteasome (prosome, macropain) | | |
| | | subunit, beta type, 3 | | |
| 22. | GLNDETYGY | 161-169 | NM_001677 | SEQ ID-No. 22 |
| | | ATPase, Na+/K+ transporting, beta | | |
| | | 1 polypeptide | | |
| 23. | GQEFIRVGY | 103-111 | NM_018154 | SEQ ID-No. 23 |
| | | anti-silencing function 1B | | |
| 24. | GQFPGHNEF | 76-84 | NM_006449 | SEQ ID-No. 24 |
| | | CDC42 effector protein (Rho | | |
| | | GTPase binding) 3 | | |
| 25. | GQPWVSVTV | 121-129 | AC005912 | SEQ ID-No. 25 |
| | | FLJ00063 | | |
| 26. | GYLHDFLKY | 254-262 | NM_012286 | SEQ ID-No. 26 |
| | | mortality factor 4 like 2 | | |
| 27. | HQITVLHVY | 137-145 | NM_021814 | SEQ ID-No. 27 |
| | | homolog of yeast long chain | | |
| | | polyunsaturated fatty acid | | |
| | | elongation enzyme 2 | | |
| 28. | HVIDVKFLY | 163-171 | NM_001923 | SEQ ID-No. 28 |
| | | damage-specific DNA binding | | |
| | | protein 1, 127 kDa | | |
| 29. | HVNDLFLQY | 484-492 | AB023222 | SEQ ID-No. 29 |
| | | KIAA1005 | | |
| 30. | IAMATVTAL | 249-257 | NM_000034 | SEQ ID-No. 30 |
| | | aldolase A, fructose-bisphosphate | | |
| 31. | IGIDLGTTY | 7-15 | NM_005345 | SEQ ID-No. 31 |
| | | heat shock 70 kDa protein 1A | | |
| 32. | ILHDDEVTV | 15-23 | NM_001003 | SEQ ID-No. 32 |
| | | ribosomal protein, large P1 | | |
| 33. | IQKESTLHL | 61-69 | | SEQ ID-No. 33 |
| | | ubiquitin A-52 residue ribosomal | | |
| | | protein fusion product 1 | | |
| 34. | ISRELYEY | 70-77 | BC022821 | SEQ ID-No. 34 |
| | | clone MCG: 39264 | | |
| | | IMAGE: 5087938 | | |
| 35. | KLHGVNINV | 59-67 | NM_002896 | SEQ ID-No. 35 |
| | | RNA binding motif protein 4 | | |
| 36. | KQMEQVAQF | 89-97 | NM_003186 | SEQ ID-No. 36 |
| | | transgelin | | |
| 37. | KVADMALHY | 296-304 | NM_006585 | SEQ ID-No. 37 |
| | | chaperonin containing TCPI, | | |
| | | subunit 8 (theta) | | |
| 38. | LEEDSAREI | 68-76 | XM_119113 | SEQ ID-No. 38 |
| | | LOC204689 | | |
| 39. | LLAERDLYL | 576-584 | NM_004613 | SEQ ID-No. 39 |
| | | transglutaminase 2 (C polypeptide, | | |
| | | protein factor 4 like 2 | | |
| 40. | LLDEEISRV | 44-52 | AB067800 | SEQ ID-No. 40 |
| | | RNA binding protein HQK-7 | | |
| 41. | LLYPTEITV | 830-838 | NM_002204 | SEQ ID-No. 41 |
| | | integrin, alpha 3 (antigen CD49C, | | |
| | | alpha 3 subunit of VLA-3 receptor) | | |
| 42. | LMDHTIPEV | 290-298 | NM_005625 | SEQ ID-No. 42 |
| | | syndecan binding protein | | |
| 43. | LQHPDVAAY | 229-237 | NM_001903 | SEQ ID-No. 43 |
| | | catenin (cadherin-associated | | |
| | | protein), alpha 1, 102 kDa | | |
| 44. | MEDIKILIA | 632-640 | NM_001530 | SEQ ID-No. 44 |
| | | hypoxia-inducible factor 1, alpha | | |
| | | subunit (basic helix-loop-helix | | |
| | | transcription factor) | | |
| 45. | MEENFAVEA | 347-355 | NM_003380 | SEQ ID-No. 45 |
| | | vimentin | | |
| 46. | MQKEITAL | 313-320 | NM_001101 | SEQ ID-No. 46 |
| | | actin, beta | | |
| 47. | NEDLRSWTA | 151-159 | NM_002127 | SEQ ID-No. 47 |
| | | HLA-G histocompatibility antigen, | | |
| | | class I, G | | |
| 48. | NEIKDSVVA | 673-681 | NM_001961 | SEQ ID-No. 48 |
| | | eukaryotic translation elongation | | |
| | | factor 2 | | |
| 49. | NVTQVRAFY | 439-447 | NM_001752 | SEQ ID-No. 49 |
| | | catalase | | |
| 50. | NYIDKVRFL | 116-124 | NM_003380 | SEQ ID-No. 50 |
| | | vimentin | | |
| 51. | PTQELGLPAY | 392-401 | NM_017827 | SEQ ID-No. 51 |
| | | seryl-tRNA synthetase 2 | | |
| 52. | QEQSFVIRA | 422-430 | NM-000211 | SEQ ID-No. 52 |
| | | integrin, beta 2 (antigen CD18 | | |
| | | (p95), lymphocyte function- | | |
| | | assocaited antigen 1; macrophage | | |
| | | antigen 1 (mac-1) beta subunit) | | |
| 53. | QQKLSRLQY | 636-644 | NM_002204 | SEQ ID-No. 53 |
| | | integrin, alpha 3 (antigen CD49C, | | |
| | | alpha 3 subunit of VLA-3 receptor) | | |
| 54. | QVAEIVSKY | 217-225 | NM_002210 | SEQ ID-No. 54 |
| | | integin, alpha V (vitronectin | | |
| | | receptor, alpha polypeptide, antigen | | |
| | | CD51) | | |
| 55. | REHAPFLVA | 30-38 | XM_208570 | SEQ ID-No. 55 |
| | | transport-secretion protein 2.2 | | |
| 56. | RLAAAAAQSVY | 5-15 | NM_000581 | SEQ ID-No. 56 |
| | | glutathione peroxidase 1 | | |
| 57. | RLASYLDKV | 90-98 | Y00503 | SEQ ID-No. 57 |
| | | keratin 19 | | |
| 58. | RNADVFLYKY | 1020-1028 | NM_007118 | SEQ ID-No. 58 |
| | | triple function domain (PTPRF | | |
| | | interacting) | | |
| 59. | RQGFVPAAY | 1012-1020 | NM_003127 | SEQ ID-No. 59 |
| | | spectrin, alpha, non-erythrocytic 1 | | |
| | | (alpha-fodrin) | | |
| 60. | RVIEEAKTAF | 198-207 | NM_002133 | SEQ ID-No. 60 |
| | | heme oxygenase (decyclingZ) 1 | | |
| 61. | RVQPKVTVY | 89-97 | AF450316 | SEQ ID-No. 61 |
| | | MHC class II antigen | | |
| 62. | RVYPEVTVY | 123-131 | L42143 | SEQ ID-No. 62 |
| | | MHC HLA-DRB1*0411 | | |
| 63. | SDHHIYL | 218-224 | NM_000034 | SEQ ID-No. 63 |
| | | aldolase A, fructose-bisphosphate | | |
| 64. | SHAILEALA | 204-212 | NM_018378 | SEQ ID-No. 64 |
| | | F-box and leucine-rich repeat | | |
| | | protein 8 | | |
| 65. | SISGVTAAY | 728-736 | NM_003870 | SEQ ID-No. 65 |
| | | IQ motif containing GTPase | | |
| | | activating protein 1 | | |
| 66. | SPVYVGRV | 216-223 | NM_004613 | SEQ ID-No. 66 |
| | | transglutaminase 2 (C polypeptide, | | |
| | | protein-glutamine-gamma- | | |
| | | glutamyltransferase) | | |
| 67. | SQFGTVTRF | 66-74 | NM_032390 | SEQ ID-No. 67 |
| | | MK167 (FHA domain) interacting | | |
| | | necleolar phosphoprotein | | |
| 68. | SWNNHSYLY | 156-164 | NM_000821 | SEQ ID-No. 68 |
| | | gamma-glutamyl carboxylase | | |
| 69. | TFMDHVLRY | 700-708 | NM_001096 | SEQ ID-No. 69 |
| | | ATP citrate lyase | | |
| 70. | TLADLVHHV | 378-386 | NM_003496 | SEQ ID-No. 70 |
| | | transformation/transcription | | |
| | | domain-associated protein | | |
| 71. | TLGALTVIDV | 1336-1345 | NM_017539 | SEQ ID-No. 71 |
| | | hypothetical protein | | |
| | | DKFZp434N074 | | |
| 72. | TQMPDPKTF | 46-54 | NM_016096 | SEQ ID-No. 72 |
| | | HSPC038 protein | | |
| 73. | VEHPSLTSP | 170-178 | M15374 | SEQ ID-No. 73 |
| | | HLA-DR beta gene, exon 2 | | |
| 74. | VEPDHFKVA | 204-212 | NM_002306 | SEQ ID-No. 74 |
| | | lectin, galactoside-binding, soluble, | | |
| | | 3 (galectin 3) | | |
| 75. | VEREVEQV | 64-71 | AI278671 | SEQ ID-No. 75 |
| | | EST reading frame +2 | | |
| 76. | VFIGTGATGATLY | 20-32 | NM_002489 | SEQ ID-No. 76 |
| | | NADH dehydrogenase | | |
| | | (ubiquinone) 1 alpha subcomplex, | | |
| | | 4, 9 kDa | | |
| 77. | VLREIAEEY | 822-830 | NM_005336 | SEQ ID-No. 77 |
| | | high density lipoprotein binding | | |
| | | protein (vigilin) | | |
| 78. | VLSLLSSVAL | 27-36 | XM_098362 | SEQ ID-No. 78 |
| | | LOC153339 | | |
| 79. | VLYDRVLKY | 484-492 | NM_014230 | SEQ ID-No. 79 |
| | | signal recognition particle 68 kDa | | |
| 80. | VMDSKIVQV | 432-440 | NM_012316 | SEQ ID-No. 80 |
| | | karyopherin alpha 6 (importin alpha | | |
| | | 7) | | |
| 81. | VQRTLMAL | 126-133 | NM_003186 | SEQ ID-No. 81 |
| | | transgelin | | |
| 82. | YFEYIEENKY | 238-247 | NM_004501 | SEQ ID-No. 82 |
| | | heterogeneous nuclear | | |
| | | ribonucleoprotein U (scaffold | | |
| | | attachment factor A) | | |
| 83. | YIFKERESF | 303-311 | NM_015947 | SEQ ID-No. 83 |
| | | CGI-18 protein | | |
| 84. | YVYEYPSRY | 164-172 | NM_006403 | SEQ ID-No. 84 |
| | | enhancer of filamentation 1 | | |
| 85. | YYRYPTGESY | 354-363 | NM_004566 | SEQ ID-No. 85 |
| | | 6-phosphofructo-2-kinase/fructose- | | |
| | | 2, 6-biphosphatase 3 | | |
| 86. | YYSNKAYQY | 230-238 | NM_024711 | SEQ ID-No. 86 |
| | | human immune associated | | |
| | | nucleotide 2 | | |
| 87. | SSLPTQLFK | 5-13 | NM_000618 | SEQ ID-No. 87 |
| | | insulin-like growth factor 1 | | |
| 88. | ATFPDTLTY | 702-710 | NM_000210 | SEQ ID-No. 88 |
| | | integrin, alpha 6 | | |
| 89. | SIFDGRVVAK | 107-116 | NM_019026 | SEQ ID-No. 89 |
| | | putative membrane protein | | |
| 90. | FRFENVNGY | 32-40 | NM_001673 | SEQ ID-No. 90 |
| | | asparagine synthetase | | |
| 91. | QRYGFSAVGF | 82-91 | NM_016321 | SEQ ID-No. 91 |
| | | Rh type C glycoprotein | | |
| 92. | AARLSLTYERL | 307-316 | NM_001183 | SEQ ID-No. 92 |
| | | ATPase, H+ transporting, | | |
| | | lysosomal interacting protecin 1 | | |
| 93. | GRYQVSWSL | 84-92 | NM_006280 | SEQ ID-No. 93 |
| | | signal sequence receptor, delta | | |
| 94. | KRFDDKYTL | 61-69 | NM_014752 | SEQ ID-No. 94 |
| | | KIAA0102 | | |
| 95. | TRWNKIVLK | 37-45 | NM_024292 | SEQ ID-No. 95 |
| | | ubiquitin-like 6 | | |
| 96. | LRFDGALNV | 242-250 | NM_006001 | SEQ ID-No. 96 |
| | | tubulin, alpha 2 | | |
| 97. | ARFSGNLLV | 310-318 | NM_013336 | SEQ ID-No. 97 |
| | | protein transport protein SEC61 | | |
| | | alpha subunit isoform 1 | | |
| 98. | NRIKFVIKR | 491-499 | NM_001518 | SEQ ID-No. 98 |
| | | general transcription factor II, I | | |
| 99. | GRVFIIKSY | 410-418 | NM_016258 | SEQ ID-No. 99 |
| | | high-glucose-regulated protein 8 | | |
| 100. | SRFGNAFHL | 538-546 | NM_006445 | SEQ ID-No. 100 |
| | | PRP8 pre-mRNA processing factor | | |
| | | 8 homolog (yeast) | | |
| 101. | GRTGGSWFK | 26-34 | NM_001677 | SEQ ID-No. 101 |
| | | ATPase, Na+K+ transporting, beta | | |
| | | 1 polypeptide | | |

1. A tumour-associated peptide with an amino acid sequence that is selected from the group consisting of SEQ ID NO: 1 to SEQ ID NO: 101, wherein the peptide has the ability to bind to a molecule of the human major-histocompatibility-complex (MHC) class-I.

2. The peptide according to claim 1, wherein at least one amino acid is replaced by a different amino acid having similar chemical properties.

3. The peptide according to claim 1, wherein N- or/and C-terminally at least one additional amino acid is present.

4. The peptide according to claim 1, wherein in that at least one amino acid is deleted.

5. The peptide according to claim 1, wherein at least one amino acid is chemically modified.

6. A method for the treatment of a tumorous disease and/or adenomatous disease, said method comprising administering to a patient in need of such treatment a peptide according to claim 1.

7. The method according to claim 6, wherein the disease is renal, breast, pancreatic, stomach, bladder and/or testes cancer.

8. The method according to claim 6, wherein the peptide is used together with an adjuvant.

9. The method according to claim 6, wherein the peptide is used bound on an antigen-presenting cell.

10. A method according to labelling of a leukocyte comprising use of a peptide according to claim 1

11. The method according to claim 10, wherein the leukocyte is a T-lymphocyte.

12. The method according to claim 10, used for determining progression of a therapy in a tumorous disease.

13. A method of producing an antibody comprising use of a peptide according to claim 1.

14. A pharmaceutical composition comprising a peptide according to claim 1.

15. A nucleic acid molecule encoding the peptide according to claim 1.

16. A method of a tumorous disease and/or adenomatous disease, said method comprising administering to a patient in need of such treatment a nucleic acid molecule according to claim 15.

17. A composition of matter comprising a nucleic acid molecule according to claim 15 selected from the group consisting of:
A. a vector comprising the nucleic acid molecule; and
B. a cell that was genetically modified with the nucleic acid molecule, such that the cell produces the peptide encoded by the nucleic acid molecule.

18. A diagnostic method wherein the presence of a peptide according to claim 1 is used as a diagnostic marker.

19. A method for the treatment of a pathological condition wherein an immune response against a protein of interest is triggered, wherein a therapeutically effective amount of a peptide according to claim 1 is administered to a patient in need of such treatment.

20. An electronic storage medium that contains the amino acid sequence of a peptide according to claim 1, and/or a nucleic acid sequence encoding the peptide.
